(19) United States
(12) Patent Application Publication
(10) Pub. No.: US 2023/0273233 A1
(43) Pub. Date: Aug. 31, 2023

(54) OPTICAL PARTICLE SENSING FOLLOWING FLUID EJECTION

(71) Applicant: Hewlett-Packard Development Company, L.P., Spring, TX (US)

(72) Inventors: Viktor Shkolnikov, Palo Alto, CA (US); Roberto A. Pugliese, Corvallis, OR (US); Jeffrey A. Nielsen, Corvallis, OR (US); Michael J. Day, Corvallis, OR (US); Diane R. Hammerstad, Corvallis, OR (US)

(73) Assignee: Hewlett-Packard Development Company, L.P., Spring, TX (US)

(21) Appl. No.: 18/015,044

(22) PCT Filed: Jul. 20, 2020
(86) PCT No.: PCT/US2020/042704

§ 371 Date: Jan. 6, 2023

(51) Int. Cl. G01N 35/10; B01L 3/02

(52) U.S. Cl. CPC G01N 35/1016; B01L 3/0268; B01L 2200/061

(57) ABSTRACT

In one example in accordance with the present disclosure, a particle dispensing system is described. The particle dispensing system includes a port to receive a number of fluid cartridges. Each fluid cartridge is to hold an amount of fluid to be ejected. The particle dispensing system also includes an optical verification system to determine, following ejection, a count of a number of particles ejected during an ejection event. The particle dispensing system also includes a controller to selectively activate a number of fluid ejectors to eject the amount of fluid.

### BACKGROUND

Cellular biology is a field of biology that studies the structure, function, and operation of cells. An understanding of the structure, function, and operation of cells provides a wealth of information. For example, individual cells may be used to generate cell lines and to aide in the further understanding of mechanisms of cellular function. As another example, once the structure, function, and operation of cells is more fully understood, certain diseases may be prevented and treated.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a particle dispensing system with optical particle sensing following fluid ejection, according to an example of the principles described herein.

FIG. 2 is a diagram of a particle dispensing system with optical particle sensing following fluid ejection, according to an example of the principles described herein.

FIG. 3 is a block diagram of a particle dispensing system with optical particle sensing following fluid ejection, according to an example of the principles described herein.

FIG. 4 is a diagram of a particle dispensing system with optical particle sensing following fluid ejection, according to an example of the principles described herein.

FIG. 5 is a diagram of a particle dispensing system with optical particle sensing following fluid ejection, according to an example of the principles described herein.

FIG. 6 is a diagram of a particle dispensing system with optical particle sensing following fluid ejection, according to an example of the principles described herein.

FIG. 7 is a diagram of a particle dispensing system with optical particle sensing following fluid ejection, according to an example of the principles described herein.

FIG. 8 is a diagram of a particle dispensing system with optical particle sensing following fluid ejection, according to an example of the principles described herein.

FIG. 9 is a diagram of a particle dispensing system with optical particle sensing following fluid ejection, according to an example of the principles described herein.

FIG. 10 is a flow chart of a method of optical particle sensing following fluid ejection, according to an example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Cellular analytics is a field of biology that uses instruments to separate, identify, and quantify matter. A wealth of information can be collected from a cellular sample. A greater understanding of the different kinds of cells and their function can lead to certain technological innovations that benefit society in countless ways. For example, from cells, certain biologics such as proteins, insulin, other therapeutic drugs, RNA, and DNA may be obtained.

Accordingly, refinements to cellular processing may enhance the possible uses and reach of cellular analytics. For example, it may be desirable to start the fabrication of materials and therapeutics from a homogeneous cell population. One way to create a homogeneous cell population is to grow the population from a single cell. Such a cell population is referred to as a clonally-derived cell line or a monoclonal cell line. That is, a clonally-derived, or monoclonal, cell line refers to cells that are derived from a single cell with well-defined properties. To generate such clonally-derived cell lines or biologics from a cell line, a scientist should be confident that the cell line was derived from a single cell.

As a specific example, a scientist may desire to generate chimeric antigen receptor (CAR) T-cells in order to combat cancerous cells in a patient. CAR T-cell therapy changes a patient's T-cells in a way that attacks the cancerous cells. Accordingly, T-cells are processed and manipulated to form CAR T-cells. Were a starting population to include an unknown quantity of T-cells, the quantity and quality of produced CAR T-cells would be reduced and/or unknown, which could affect the efficacy of a session of the CAR T-cell therapy.

Accordingly, by using a starting population for which the characteristics are verified, confidence and reliability in the subsequent operations is promoted. In other words, as can be imagined, uncertainty regarding the characteristics of a starting population introduces uncertainty and inaccuracy in an output and for any operation subsequently carried out, whether that operation be analysis to identify a particular relationship, cell generation, and/or biologic generation. Therefore, it may be desirable to separate single cells into growth chambers, and to verify that there was a single cell in the chamber, and not multiple cells as having multiple cells will likely increase the heterogeneity of the progeny population.

While some devices may perform cell sorting, such systems are ineffective for a number of reasons. For example, in some cases cells may be sorted by fluorescence activated cell sorting (FACS) and magnetically activated cell sorting (MACS), both of which rely on labels to identify the cells, a single cell at a time. However, the presence of such labels may interfere with cellular operation and analysis of cell functionalities. Accordingly, the present specification provides for such sorting and singulation without labeling agents, thus preserving the integrity of any subsequent operations.

Label-free singulation of cells may involve expensive systems that incorporate video detection integrated with dispensing, or dilution of the cell suspension, and portioning the suspension into individual volumes (wells). One specific example is using a limiting dilution. In a limiting dilution, a cell suspension is diluted such that the number density (i.e., the number of cells/the number of chambers) is low, such that that number of cells in a well follows a Poisson distribution. However, in limiting dilution, many of the wells may be wasted (increasing cost), while a number of the non-empty wells contain 2 or more cells. Moreover, a scientist may not know which wells are empty, which have multiple cells, and which have the desired single cell. This uncertainty in the initial sample of cells propagates to uncertainty regarding any analyzed output and may add heterogeneity into the resulting population.

In some examples, a cell solution is loaded into an ejection system and cells are ejected one at a time by observing the presence of a cell in a chamber upstream of a fluid ejector. While this may reduce the number of empty wells, cells may become stuck in the dispense orifice, accumulate there, and then several cells may be ejected into a single chamber, resulting in a non-homogenous population. That is, even though cells are positioned to be ejected one at a time, for any number of reasons more than one cell may be ejected, or no cells may be included in the portion of fluid ejected during an ejection event.

Accordingly, the present specification describes a particle dispensing system with optical verification. The system controls a fluid ejection device and an optical verification system which observes either 1) the target substrate, i.e., wells of the well plate or 2) the droplet right before hitting the target substrate to verify the number of cells (or particles) that are to be located at the target location.

That is, the present system monitors the cells downstream of the dispense orifice. Such monitoring ensures 1) that the desired number of cells (e.g. a single cell) are actually dispensed into the wells and 2) that the resulting population is monoclonal. The present specification generates a well-defined starting population which ensures that single cells are used to generate a clonally-derived cell line. Accordingly, there can be greater confidence in the outcome of any subsequent operation. Continuing the above example, T-cells may be placed in individual wells such that a user may know with certainty what cells and what quantity of cells are present in a starting population such that CAR T-cells of a determined quantity may be formed. That is, the present specification reduces the unknowns from a starting population such that a scientist may more accurately carry out subsequent chemical operations.

Specifically, the present specification describes a particle dispensing system. The particle dispensing system includes a port to receive number of fluid cartridges, each fluid cartridge to hold an amount of fluid, which fluid includes particles to be ejected. The particle dispensing system also includes an optical verification system to determine, following ejection, a count of a number of particles ejected during an ejection event. The particle dispensing system also includes a controller to selectively activate a number of fluid ejectors to eject the amount of fluid.

In an example, the particles within the fluid comprise cells to be ejected. In some examples, the optical verification system is selected from the group consisting of 1) a camera and 2) a light source to emit light and a detector to capture scattered light from the light source. The particle dispensing system may include a database to map detected scattered light to the count of the number of particles.

In some examples, the optical verification system is directed downward towards a substrate on which the fluid is deposited. In another example, the optical verification system is directed upward through a substrate on which the fluid is deposited. In yet another example, the optical verification system is directed across a path of the fluid as it falls onto the substrate.

The present specification also describes a method. According to the method, a fluid ejector of a particle dispensing system is activated to eject a cell onto a substrate. The fluid is optically detected following ejection. Following ejection, an optical verification system determines whether a desired quantity of cells was ejected during an ejection event.

In an example, determining whether the desired quantity of cells was ejected during the ejection event is triggered by an ejection monitoring system indicating uncertainty regarding a quantity of cells ejected. In some examples, optically detecting the fluid following ejection includes optically detecting the fluid as it falls. In another example, optically detecting the fluid following ejection includes optically detecting the fluid on the substrate.

In an example, as fluid is being deposited at a first location of the substrate, an optical verification system is determining whether a single cell was ejected at a second location previously dispensed onto.

The present specification also describes a particle dispensing system. In this example, the particle dispensing system includes a port to receive a number of fluid cartridges, each fluid cartridge to hold cells to be ejected individually during an ejection event. The particle dispensing system also includes an optical verification system to, following ejection, verify that a single cell was ejected during the ejection event. The particle dispensing system also includes a controller to selectively activate a number of fluid ejectors to eject the amount of fluid. In this example, the particle dispensing system includes a stage controller to suppress a motion of a stage holding a substrate during optical verification.

In an example, the optical verification system includes multiple optical verification devices to verify that a single cell was ejected during the ejection event. In an example, the particle system further includes the stage to hold the substrate, wherein the stage allows optical verification from underneath the substrate.

Note that while the present specification describes cells as a particular type of target particle, the present systems and methods may target and eject other types of particles including beads of various materials such as metal and latex, DNA-functionalized beads, and other microspheres. That is, while target particles may be of a wide-variety of types, in one specific examples, the particles are cells.

Note that throughout the specification, while specific reference is made to deposition of fluid into wells of a well-plate, the present systems and devices can be used to deposit fluid on other target surfaces such as microscope slides, matrix assisted laser desorption/ionization (MALDI) plates, petri dishes, and microfluidic chips among other substrates or surfaces. In the later examples, the substrate may include a fixture that fits into the corner ribs and the clamp and retains the target surface.

In summary, using such a particle dispensing system 1) provides highly accurate cell separation; 2) is low cost; 3) provides for the rapid generation of many singulated cells; 4) avoids additional labeling reagents; and 5) avoids separate verification tools/operations. However, the devices disclosed herein may address other matters and deficiencies in a number of technical areas.

As used in the present specification and in the appended claims, the term “fluidic die” refers to a component of a particle dispensing system that ejects fluid and includes a number of fluid ejectors.

Accordingly, as used in the present specification and in the appended claims, the term “fluid ejector” refers to an individual component of a fluidic die that ejects fluid.

Accordingly, as used in the present specification and in the appended claims, the term “fluid ejection device” refers to a fluidic die and a reservoir associated with the fluidic die.

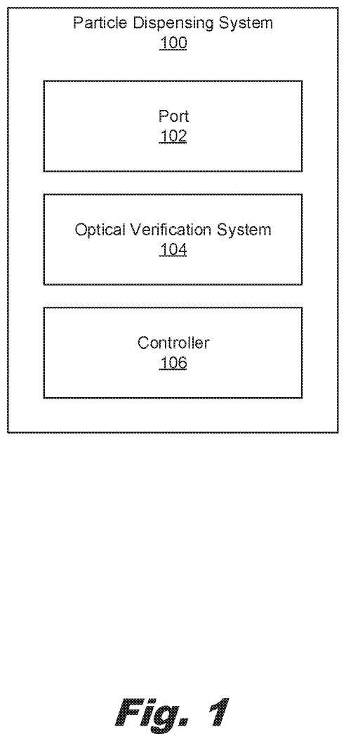

Turning now to the figures, FIG. 1 is a block diagram of a particle dispensing system (100) with optical particle sensing following fluid ejection, according to an example of the principles described herein. In some examples, the particle dispensing system (100) may be a microfluidic structure. In other words, the components, i.e., the fluid ejectors (106) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

As described above, such particle dispensing systems (100) are used to deposit fluid onto a substrate. The substrate may be any material on which fluid may be dispensed. In one example, the substrate may be a well-plate with a number of wells in an array and the fluid may be deposited into the individual wells of the well-plate. While specific reference is made to deposition of fluid into wells of a well-plate, the present systems and devices can be used to deposit fluid on other substrates or surfaces such as microscope slides, matrix assisted laser desorption/ionization (MALDI) plates, and microfluidic chips among other substrates or surfaces.

A variety of fluids may be deposited. For example, the fluid ejection system may be implemented in a laboratory and may eject biological fluid. The fluid dispensed by the fluid ejection system may be of a variety of types and may be used for a variety of applications. In some examples, the biological fluid may include solvent or aqueous-based pharmaceutical compounds, as well as aqueous-based biomolecules including proteins, enzymes, lipids, antibiotics, mastermix, primer, DNA samples, cells, or blood components, all with or without additives, such as surfactants or glycerol. To eject the fluid, the controller (106) passes control signals and routes them to fluid ejectors of the fluid ejection system.

As described above, the particle dispensing system (100) receives fluid cartridges and includes an optical verification system (104) which observes the system post-ejection. That is, the optical verification system (104) analyzes either the wells of the well plate or the droplets of fluid after ejection but before entering the well plate to verify the number of cells (or particles) that are ejected into the well of interest. Such an optical verification system (104) may be implemented in many forms. For example, the optical verification system (104) may use a through-meniscus imaging camera. In a second example, the optical verification system (104) images through the bottom of the substrate to locate and count the cells directly. In yet another example, light-scattering is used to observe the cells in the droplet about to enter the well, or in a well directly.

To carry out particle dispensing, the particle dispensing system (100) includes a variety of sub-components. Specifically, the particle dispensing system (100) includes a port (102) to receive a number of fluid cartridges. That is, the fluid cartridges may be removable from the particle dispensing system (100). Accordingly, a port (102) may include an opening into which the fluid cartridge is inserted. In some examples, the port (102) receives multiple fluid cartridges which simultaneously eject fluid onto multiple locations of a target substrate. In another example, the port (102) receives a single fluid cartridge. Accordingly, the port (102) may be sized to receive one or multiple fluid cartridges. The port (102) may include a mechanism to securely hold the fluid cartridges. The port (102) may also include electrical contacts which mate with corresponding contacts on the fluid cartridges. That is, as described above, the controller (106) may operate to selectively eject fluid from the fluid cartridges. The signal by which the controller (106) activates the fluid ejection is transmitted from the controller (106) to the fluid cartridges via these electrical contacts.

The fluid cartridges may take a variety of forms. For example, the fluid cartridge may be a fluid ejection device. A fluid ejection device includes a reservoir to hold an amount of fluid to be ejected. In some examples, the reservoir may be open such that a user may manually insert a sample with target cells into the reservoir, for example via a pipette. As described above, the fluid may include a carrier fluid and may also contain cells or other particles that are to be individually ejected. That is, the cells or particles may be suspended in a variety of appropriate fluids. One example of a fluid in which cells are suspended includes a solution of Phosphate Buffered Saline (PBS). Another example may be a solution of cell growth media matched to the cell type.

Each fluid ejection device may also include a fluid ejector to eject the amount of fluid, and corresponding particle, from the reservoir. That is, the particle dispensing system (100) may hold the fluid ejection devices above a surface onto which a target particle is to be ejected. As a specific example, the particle dispensing system (100) may form part of a fluid analysis system that includes a stage to hold a well plate. It may be desirable to dispense target particles into individual wells of the well plate. Accordingly, the fluid ejection devices include fluid ejectors that expel the target particles through openings towards the individual wells of the well plate.

The fluid ejector may include a firing resistor or other thermal device, a piezoelectric element, or other mechanism for ejecting particles from the firing chamber. For example, the fluid ejector may be a firing resistor. The firing resistor heats up in response to an applied voltage. As the firing resistor heats up, a portion of the fluid adjacent the firing resistor vaporizes to form a bubble. This bubble pushes the cell to be analyzed out an orifice and onto a surface such as a micro-well plate. As the vaporized fluid bubble collapses, a vacuum pressure along with capillary force draws additional fluid towards the fluid ejector, and the process repeats. In this example, the fluid ejector may be a thermal inkjet fluid ejector.

In another example, the fluid ejector may be a piezoelectric device. As a voltage is applied, the piezoelectric device changes shape which generates a pressure pulse that pushes a fluid out the orifice. In this example, the fluid ejector may be a piezoelectric inkjet fluid ejector.

In another example, the port (102) receives a fluid cartridge that is pre-filled with a fluid that contains particles. In this example, the particle dispensing system (100) may count and identify the particles as they exit. For example, the fluid cartridge may be a cassette with fluid therein. As a specific example the cassette may include a blister pack with fluid. Accordingly, the fluid cartridge contains fluid before it is secured to the particle dispensing system (100). In this example, the fluid ejector may be formed on the pre-filled container or the fluid ejector may be a part of the particle dispensing system (100) that interacts with the inserted container, i.e., the inserted blister pack. In another example, such a pre-filled fluid cartridge may include an inkjet type cartridge, for example that may be shipped with dyna beads where a certain number of beads are to be ejected into a well. That is, any pre-filled fluid cartridge may be any fluid cartridge that contains fluid at some point may be secured into the particle dispensing system (100).

The particle dispensing system (100) also includes an optical verification system (104) to determine a count of a number of particles ejected during an ejection event. In one specific example, the optical verification system (104) may verify whether a single cell was ejected during the ejection event. That is, the fluid ejector may be designated to eject a single cell during an ejection event. However, for any variety of reasons, it may be the case that more than one cell is ejected.

For example, a firing pulse may be strong enough that multiple cells are ejected together. As another example, it may be the case that two cells have become stuck together such that although a single ejection event is triggered, multiple cells are ejected. As yet another example, it may be the case that control signals to the fluid ejector are altered inadvertently or due to mechanical and/or electrical interference such that multiple ejection events occur when a single ejection event was intended.

In yet another example, it may be the case that for a particular portion of the fluid being ejected, no cells are present. Accordingly, despite a single ejection event being triggered, it may be that just the carrier fluid and no cell is deposited at a target location on the surface.

As such, the optical verification system (104) operates to determine how many cells have been ejected. Note that this is a post-ejection measurement. That is, the optical verification system (104) does not predict that a single cell will be ejected, for example via a sensor upstream of the fluid ejector. Rather the optical verification system (104) determines, following ejection, what was actually ejected.

The optical verification system (104) may take many forms and may be positioned at different locations on the particle dispensing system (100). As one particular example, the optical verification system (104) is a camera that captures an image of the fluid as it drops or as it sits at the target location. Such a camera may detect or otherwise image cells found in the droplets of fluid that are ejected. As a specific example, the camera may include a complimentary metal-oxide-semiconductor (CMOS) or charge-coupled device (CCD) imaging array and magnification optics to project an image of the cell and its surroundings onto the imaging array. The image is then processed via, for example, blob detection to detect the particle and characterize its features. It's size and curvature features can then be used (via a database) to classify it as a single cell vs. debris or multicell clumps.

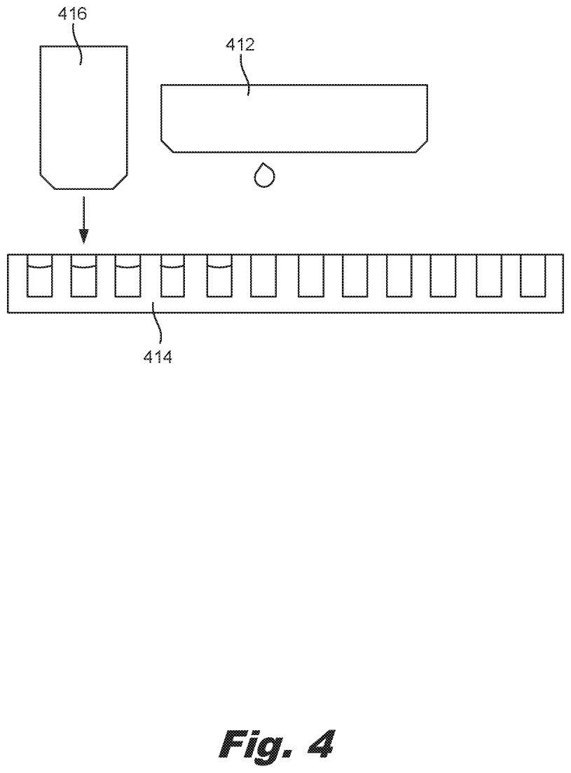

In another example, the optical verification system (104) includes a light source and a detector. As a cell passes through the light generated by the light source, the light is scattered. The scattered light is detected by a detector and mapped to determine whether one or multiple particles have passed through the light stream. That is, the scattered light signal may be classified as coming from a single cell or a multicell clump. In summary, the optical verification system (104) includes hardware components that are downstream of a fluid ejector and which can detect whether a single cell has been ejected. FIGS. 4-9 depict various examples of types and orientations for the optical verification system (104).

The particle dispensing system (100) may include a controller (106) to selectively activate the number of fluid ejectors to eject the amount of fluid. That is, the controller (106) may periodically send control signals through electrical contacts in the port (102). These signals activate the fluid ejectors. For example, these signals may heat up the resistors such that they operate to eject fluid.

The controller (106) may also receive and transmit data collected by the optical verification system (104). That is, the controller (106) may receive image data from the camera and measurements from a detector. In some cases, the controller (106) may pass this information onto a computing device for analysis, or may perform the analysis itself.

The controller (106) may include various hardware components, which may include a processor and memory. The processor may include the hardware architecture to retrieve executable code from the memory and execute the executable code. As specific examples, the controller as described herein may include computer readable storage medium, computer readable storage medium and a processor, an application specific integrated circuit (ASIC), a semiconductor-based microprocessor, a central processing unit (CPU), and a field-programmable gate array (FPGA), and/or other hardware device.

The memory may include a computer-readable storage medium, which computer-readable storage medium may contain, or store computer usable program code for use by or in connection with an instruction execution system, apparatus, or device. The memory may take many types of memory including volatile and non-volatile memory. For example, the memory may include Random Access Memory (RAM), Read Only Memory (ROM), optical memory disks, and magnetic disks, among others. The executable code may, when executed by the controller (106) cause the controller (106) to implement at least the functionality of selectively activating the number of fluid ejectors as described below.

The optical verification system (104) may be integrated on the same device that holds the fluid cartridge. That is, rather than removing a substrate from a fluid analysis system that includes the fluid cartridge, the particle dispensing system (100) provides an in-situ cell ejection verification system.

An example of fluid flow through the particle dispensing system (100) is now described. In this example, a sample which includes T-cells and a carrier fluid is held in a reservoir of a fluid cartridge. The fluid cartridge is placed in the port (102) where it is mechanically held in place and where electrical contact is established with the controller (106). The controller (106) sends control signals activate the fluid ejectors such that a portion of the fluid is passed into a well of a titration plate. Either as the fluid falls, or after it has been deposited into a well, the optical verification system (104) analyzes the fluid to determine if a single T-cell was ejected. If so, the particle dispensing system (100) may generate an output which is passed to a computing device indicating successful single-cell ejection in that well. By comparison, if more than one T-cell or no T-cell was ejected, the particle dispensing system (100) may generate an output indicating the contents of that well, i.e., no cell or multiple cells.

Other examples of the cells of interest that are included in the sample and that may be the subject of the optical verification system (104) include the NCI-60 human tumor cells including MDA-MB-231, MDA-MB-468, 5B1, 8988T PDAC, Vero E6 and A549 cells. Other examples include Chinese hamster ovary (CHO) cells, 3T3 cells, human umbilical vein endothelial (HUVAC) cells, EL-4 cells, human embryonic kidney (HEK) 293 cells, jurkat cells, and HeLa cells to name a few. While a few specific cells from which cell lines may be derived are mentioned, other cells may be similarly dispensed via the particle dispensing system (100) described herein.

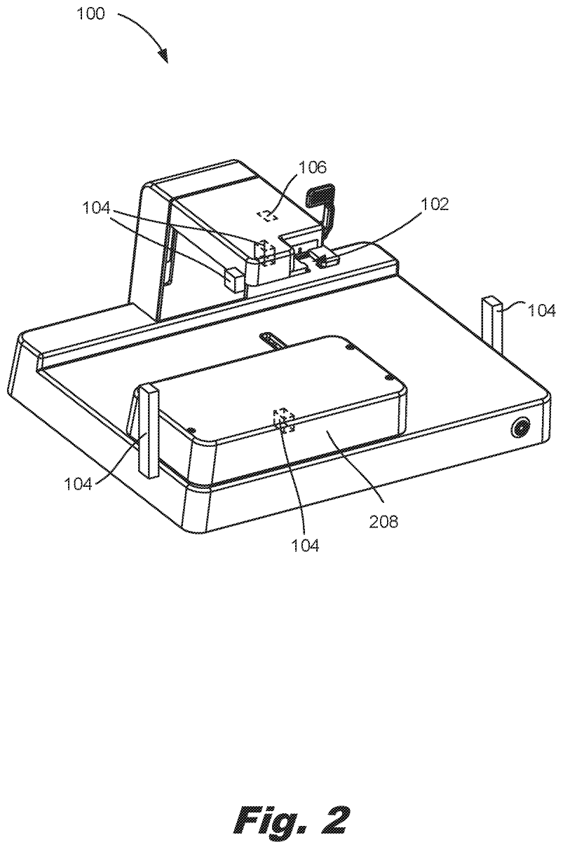

FIG. 2 is a diagram of a particle dispensing system (100) with optical particle sensing following fluid ejection, according to an example of the principles described herein. In some examples, the particle dispensing system (100) may a benchtop device. For example, the particle dispensing system (100) may be positioned on top of a table in a laboratory. In another example, the particle dispensing system (100) may include a conveyance system. In this example, the conveyance system may move the substrate stage (208) with the associated substrate to different locations within a room wherein different operations may be performed at the different stages.

As described above, the particle dispensing system (100) may include a port (102) to receive a fluid cartridge, whether that fluid cartridge be a fluidic ejection device to be filled with a fluid or a pre-filled cassette. That is, in this example the fluid cartridge is removable from the particle dispensing system (100).

In this example, the particle dispensing system (100) may include a vertical support and a head coupled to the vertical support. The port (102) may be disposed on the head. In some examples the head may move using any mechanism including, for example, a number of mating rails with one half of the mating rails being coupled to the vertical support and the other half of the mating rails being coupled to the interface. Such a benchtop particle dispensing system (100) may also include a base to hold the vertical support.

The particle dispensing system (100) may also include a stage (208) to hold the substrate. In some examples, such as when the optical verification system (104) is disposed underneath the substrate, the stage (208) allows for optical verification from underneath the substrate. As a particular example, the stage (208) may be optically transparent. In another example, the stage (208) may include a cutout underneath the target locations. For example, a frame-type stage (208) may expose the underside of the wells such that the optical verification system (104) may image the contents of the well.

The stage may be movably coupled to the base. In this example, the stage (208) may move as instructed by the controller (106) in order to place the substrate into a desired position underneath the fluid cartridge which is disposed within the port (102). In another example, the stage (208) may be stationary. For example, the stage (208) may include a vibration isolation table.

FIG. 2 also depicts example locations for the optical verification system (104). For example, when the optical verification system (104) images downward onto the substrate or perpendicular to the flow path of the fluid towards the substrate, the optical verification system (104) may be disposed on the head, near the port (102). By comparison, when the optical verification system (104) images through the substrate, the optical verification system (104) may be disposed in or underneath the stage (208).

While FIG. 2 depicts various instances of the optical verification system (104), the particle dispensing system (100) may include one optical verification system (104) disposed at any of the locations depicted in FIG. 2. Moreover, while FIG. 2 depicts certain locations for the optical verification system (104), the optical verification system (104) may be positioned at other location.

FIG. 2 also symbolically represents the controller (106) which controls ejection of the fluid, manages operation of the optical verification system (104), and controls the movement of the stage (208). As described above the controller (104) may also generate the feedback that is presented to the user regarding a quantity of cells that have been deposited on the substrate.

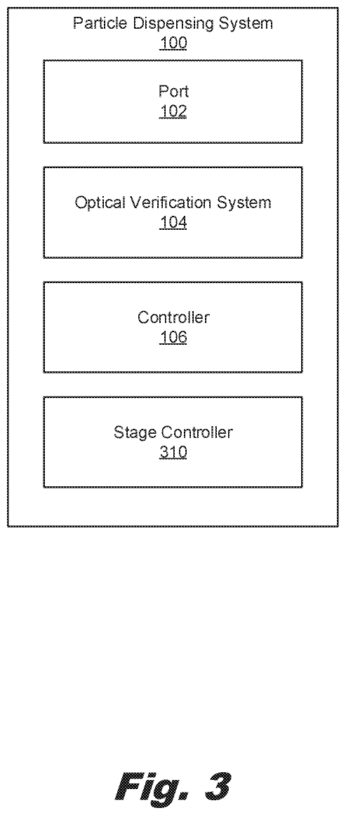

FIG. 3 is a block diagram of a particle dispensing system (100) with optical particle sensing following fluid ejection, according to an example of the principles described herein. As described above, the particle dispensing system (100) includes a port (102) to receive a number of fluid cartridges, an optical verification system (104) to verify that a desired quantity of cells was ejected during an ejection event, and a controller (106) to selectively activate the number of fluid ejectors. In the example depicted in FIG. 3, the particle dispensing system (100) further includes a stage controller (310) to suppress a motion of the stage (FIG. 2, 208) during optical verification.

That is, in some examples, the optical verification system (104) may image through the fluid to determine the presence of a single cell. For example, as depicted in FIG. 4, the optical verification system (104) may capture an image through a surface of the fluid. In certain target substrates, such as well plates, a meniscus may form on the top of the fluid. If this meniscus moves during imaging, the optical verification system (104) may incorrectly indicate whether or not a single cell was ejected. In other cases, the optical verification system (104) may not be able to accurately measure at all due to the motion of the meniscus. That is, while the shape of the meniscus may be accounted for and accurate images may be generated even through the meniscus, the motion of the meniscus may complicate, or may even prohibit image capture through the fluid.

Accordingly, in this example, the particle dispensing system (100) includes a stage controller (310) to stop the vibration of the meniscus, reduce the vibration of the meniscus, or counter the vibrations computationally. That is, during operation the stage (FIG. 2, 208) may move to align different target locations (e.g., wells) underneath the fluid cartridge(s). As the stage moves, fluid that is already deposited in wells may oscillate. Moreover, the motors that move the stage (FIG. 2, 208) may cause the stage (FIG. 2, 208) to vibrate, which vibration transfers to the fluid in the wells. Put another way, stage (FIG. 2, 208) motors have jitter that result in vibrations that shake the stage (FIG. 2, 208) and the meniscus, and therefore distort the image. The stage controller (310) operates to prevent the distortion that fluid motion and/or motor vibration introduce.

In one example, this may include reducing the speed of motion of the stage (FIG. 2, 208) so that any resulting oscillations are within a certain range where the optical verification system (104) may still carry out accurate measurements. In another example, the stage controller (310) may provide less current to the motors to reduce motor-induced vibrations. In another example, the power to the motor may be reduced or entirely shut off during optical verification. That is, the stage controller (310) may move the stage (FIG. 2, 208) to align a new target location with a fluid cartridge. The stage controller (310) may then turn off the motor, or reduce the power to the motor during optical verification and/or fluid ejection to ensure measurement accuracy. Once optical verification is complete, power may be restored such that the stage (FIG. 2, 208) may again be moved and fluid ejected towards a new target location.

In any of these examples, the stage controller (310) may include an accelerometer to measure the vibrations/movement of the stage (FIG. 2, 208). As described above, if accelerometer output is beyond a certain threshold, i.e., a threshold wherein resulting motions at the meniscus would distort images and/or prohibit accurate imaging, the stage controller (310) may operate as described above to reduce the vibrations and oscillations.

FIG. 4 is a diagram of a particle dispensing system (FIG. 1, 100) with optical particle sensing following fluid ejection, according to an example of the principles described herein. Specifically, FIG. 4 depicts an example of the optical verification system (FIG. 1, 104) which includes an imaging camera (416) that takes a picture or otherwise generates an image of the fluid in the well and thereby detects whether the well includes a cell. As described above, a CMOS or CCD imaging array projects an image of the cell and its surroundings onto the imaging array. The image is then processed via, for example, blob detection to detect the particle and characterize its features.

In some examples, the camera (416) may be a brightfield system, a phase contrast system, a differential interference contrast system, or a fluorescence system. The optical verification system (FIG. 1, 104) may include an illumination source to enhance the clarity in the resulting images. Such an illumination source may implement epi illumination, omni illumination (e.g., room lights), and/or a collimated beam (e.g., a laser).

In an example, to verify the number of cells in a well after an ejection event, the optical verification system (FIG. 1, 104) images the well plate directly. As described above, the resulting image is analyzed computationally for presence of objects, and the objects are identified as either a cell, multiple cells, or not a cell and are counted. In some examples, the results are output to a user, i.e., whether the well includes a single, multiple, or no cells. In some examples the output may be a mapping between wells and their contents. In some examples, an image is transmitted to the user.

The optical verification system (FIG. 1, 104) may be oriented in different ways. In the example depicted in FIG. 4, the optical verification system (FIG. 1, 104), e.g., the camera (416), is directed downwards onto the substrate (414), which in this example is a well plate. In this example, the camera (418) images through the meniscus of the fluid and the camera (416), or the controller (FIG. 1, 106) may compensate for the curvature of the meniscus to generate an accurate image of the contents of the well. That is, the meniscus curvature bends light and so has an optical transfer function, taking an input image matrix and producing an output image matrix. The controller (FIG. 1, 106) may compensate for this transfer function by finding its inverse applying it to output matrix to obtain the original input matrix.

As depicted in FIG. 4, the particle dispensing system (100) may image one location on the target surface while the particle dispensing system (100) ejects fluid at another location. That is, the fluid ejector (412) may eject fluid into one well while the camera (416) images another well. Doing so may increase the efficiency as the stage (FIG. 2, 210) does not need to move following each ejection event to verify single-particle ejection. Rather, the stage (FIG. 2, 208) may move the substrate (414) such that different target locations receive the fluid and as the target locations pass under the camera (416), they are imaged to determine whether a desired quantity of cells was ejected into that well. The output may then be recorded and passed to a user.

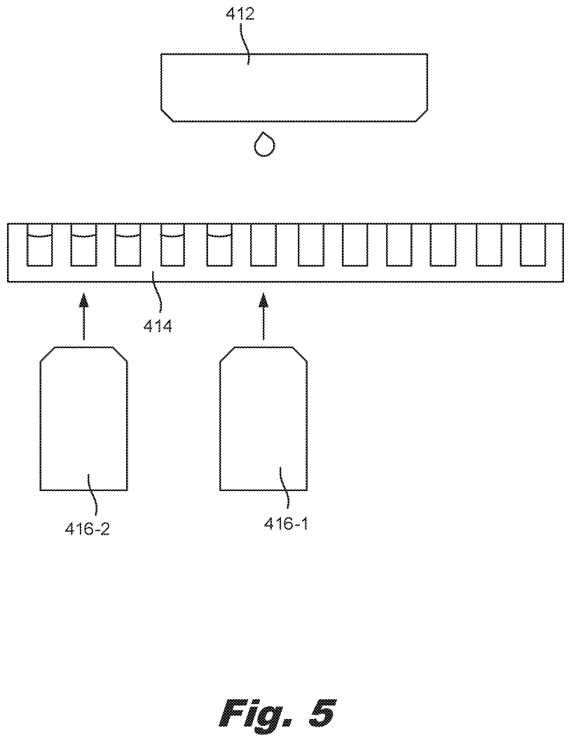

FIG. 5 is a diagram of a particle dispensing system (FIG. 1, 100) with optical particle sensing following fluid ejection, according to an example of the principles described herein. Specifically, FIG. 5 depicts an example where the optical verification system (FIG. 1, 104) includes multiple optical verification devices and wherein the optical verification devices are disposed under the substrate (414).

With the optical verification system (FIG. 1, 104) being disposed underneath the substrate (414), the cameras (416-1, 416-2) do not image through the meniscus such that motion of the fluid from oscillating waves and/or motor-induced vibration has a reduced impact on image distortion and/or the ability of the cameras (416) to generate accurate images.

In this example, where the optical verification system (FIG. 1, 104) is disposed underneath the substrate (416) and as such is directed upward through the substrate, the substrate stage (FIG. 2, 208) may allow for optical access of the cameras (416) to the substrate (414). In one example, the stage (FIG. 2, 208) may be optically transparent. In another example, the stage (FIG. 2, 208) may include an opening through which the cameras (416) or other optical verification system (FIG. 1, 104) may view and image the fluid on the substrate (414).

FIG. 5 also depicts different locations where the cameras (416) may be placed. For example, a camera (416-1) may be disposed directly under the fluid ejector (412) such that the well is imaged in real time as fluid is being ejected towards that well. That is, as the fluid ejector (412) ejects fluid into a well, the first camera (416-1), which is directly under that well, may image to determine whether a cell has been deposited in that well. This may result in increased efficiency as a well both receives a cell and is imaged at the same time. In one particular example, the fluid ejector (412) and the optical verification system (FIG. 1, 104) are rigidly connected to one another, such as by a C-shaped bracket, to enable relative motion of the stage (FIG. 2, 208) and substrate (414) between them, while maintaining alignment between these two components.

In another example, as described above in connection with FIG. 4, the camera (416) may be off-center from the fluid ejector (412). In this example, the fluid ejector (412) may be ejecting fluid into one well while the second camera (416-2) is imaging another well. As in the previous example, the imaging and fluid ejection may occur simultaneously, albeit at different locations.

FIG. 5 also depicts an example where the optical verification system (FIG. 1, 104) includes multiple optical verification devices. In this case, the cameras (416-1, 416-2) to verify that a single cell was ejected during the ejection event. That is, an output from both of the cameras (416-1, 416-2) may be received and compared to one another. The controller (FIG. 1, 106) may determine that a single cell was ejected into a well when both outputs so indicate. If one camera (416-1) output indicates that a single cell was ejected into a well, but another camera (416-2) output indicates that no cell or multiple cells were ejected into the well, the controller (FIG. 1, 106) may record a singularity and present a notification to the user. Such a multi-verification device system provides even more assurance regarding the count of cells ejected towards a target location.

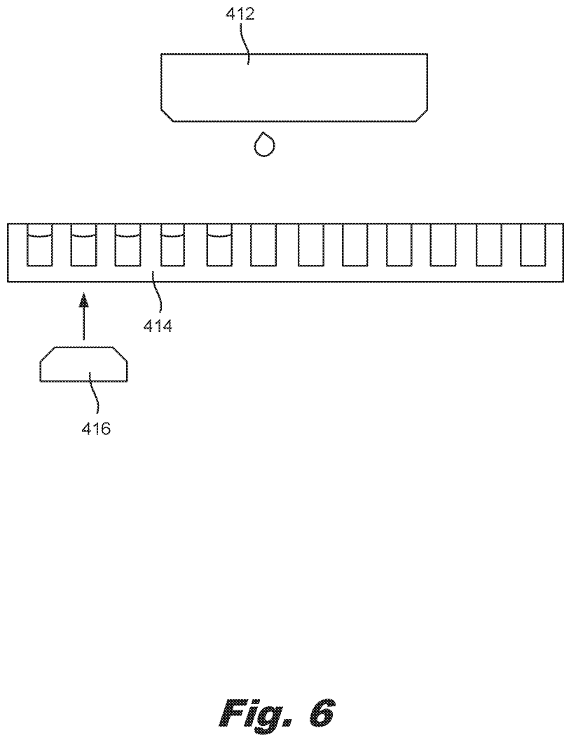

FIG. 6 is a diagram of a particle dispensing system (FIG. 1, 100) with optical particle sensing following fluid ejection, according to an example of the principles described herein. In the example depicted in FIG. 6, the camera (416) is disposed underneath the substrate (414) and is directed upward through the substrate (414). In some examples, the camera (416) may include flat optic lenses. Examples of flat optic lenses include Fresnel lenses and a gradient-index (GRIN) optic lens. Such lenses may allow for the camera (416) to be smaller, thus conserving space in an otherwise crowded fluid ejection system. Fresnel lenses and GRIN optic lenses may be lower weight such that if attached to the stage (FIG. 2, 208), the stage (FIG. 2, 208) may have less inertia and may allow for faster stage (FIG. 2, 208) movement.

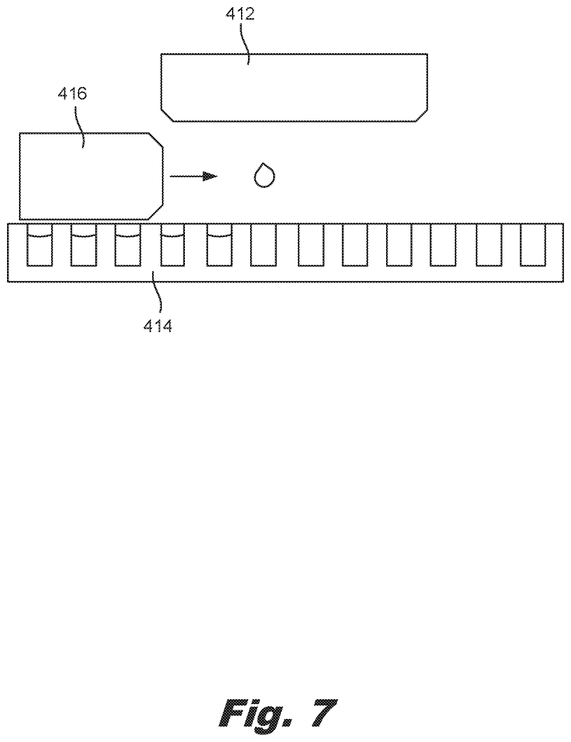

While FIG. 6 depicts such a flat optic camera (416) in a below-mounted optical verification system (FIG. 1, 104), such a flat optic camera (416) may be mounted in another arrangement, such as an above-mounted optical verification system (FIG. 1, 104) as depicted in FIG. 5 or a side-mounted optical verification system (FIG. 1, 104) as depicted in FIG. 7.

FIG. 7 is a diagram of a particle dispensing system (FIG. 1, 100) with optical particle sensing following fluid ejection, according to an example of the principles described herein. In the example depicted in FIG. 7, the optical verification system (FIG. 1, 104), i.e., the camera (416), is directed across a path of the fluid as it falls onto the substrate (414). That is, as the fluid droplet falls from the fluid ejector (412), the camera (416) images the droplet to determine whether it contains a cell.

In this example, the droplet is imaged before it enters the well, to ensure that the droplet or droplets destined for the well actually contain the desired number of cells. Doing so may simplify the cell sensing operation as no computational corrections are used to offset imaging through a transparent stage (FIG. 2, 208) or the meniscus of the well. In such an example, the controller (FIG. 1, 106) may synchronize the image capture by the camera (416) to the fluidic ejection so that, for example, the droplet enters the frame at the same position. This may be referred to as stroboscopic or pseudo stroboscopic imaging.

Specifically, a database may indicate the time between the firing pulse and the appearance of a droplet with particles at the imaging location. This timing may be a function of firing parameters and the dispensing die geometry. The controller (FIG. 1, 106) would use this timing to operate the camera (416). This database may also include the particle velocity at the imaging region, as to inform when to take subsequent exposures, potentially on the same frame. The controller (FIG. 1, 106) may use this information to gate the exposure of the camera and/or the illumination source.

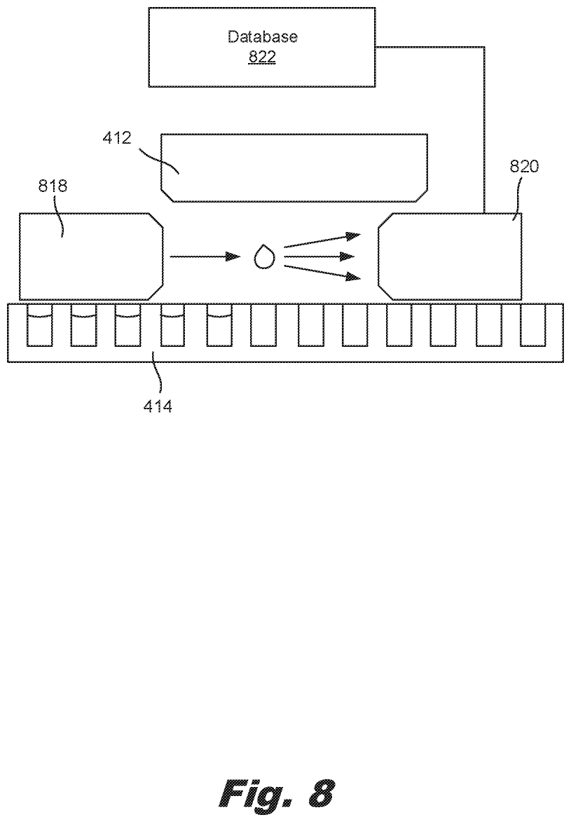

FIG. 8 is a diagram of a particle dispensing system (FIG. 1, 104) with optical particle sensing following fluid ejection, according to an example of the principles described herein. As described above, the optical verification system (FIG. 1, 104) may take many forms. In the example depicted in FIG. 8, the optical verification system (FIG. 1, 104) includes a light source (818) that emits light and a detector (822) which detects the light scattered as it passes through a fluid droplet.

That is, as the light hits a droplet, some of the light scatters in different directions. The detector (820) collects the scattered light and can determine certain attributes of the scattered light such as the scatter angle. In a specific example, the detector (820) may include an array of photosensitive elements (e.g. avalanche photo diode array) arranged with a predetermined spacing and angle to each other. Scattered light intensity is measured at each element, the light intensity at each location representing the light scattering signature of the particle. This would be correlated to the particle type, and used with the database (822) to correlate between detector signal and particle type.

In a more specific example, the detector (820) includes a series of concentric photosensitive elements that are centered on the beam of a laser in the case of laser diffraction. When ejected drops are of variable size, size measurement may be implemented as follows. Light not diffracted by particles/drops hit the center photosensitive element, light hitting large drops are diffracted to a first degree and hit photosensitive elements on inner rings of photosensitive elements. Light hitting small drops are diffracted to a second degree and hit photosensitive elements on outer rings. Accordingly, when drops are of a predetermined size, then the diffraction amount can be correlated to whether drops had none, one, or more than one particle.

In summary, different cells, and more particularly different quantities of cells, scatter the light differently. The detector (820) can differentiate single cell vs. multi-cell ejection based on these different properties. In some cases, the detector (820) can identify the cell based on the different properties. Accordingly, the particle dispensing system (FIG. 1, 100) may determine from the properties of the detected scattered light whether or not a cell, or how many cells, have been ejected.

Accordingly, the particle dispensing system (FIG. 1, 100) may include a database (822) that maps detected scattered light to a count of a number of cells. That is, the detector (820) may detect scattered light and an output may be compared to entries in the database (822) to determine whether the output indicates that a single cell was ejected, whether no cell was ejected, or whether multiple cells were ejected. In some examples, the mapping between the light scattering from the droplet and the content of the droplet may be achieved with, for example, a neural network. That is, as each measurement is made and a cell count determined therefrom, the data may be added to a database and a machine-learning system may refine a model such that more accurate assessments regarding single-cell ejection may be made.

The light source (818) may be of a variety of types including a laser, or collimated light. The detector (820) may also be of a variety of types including a charge-coupled device (CCD), a complimentary metal-oxide-semiconductor (CMOS) array, or a point detector such as a photomultiplier tube or an avalanche photodiode.

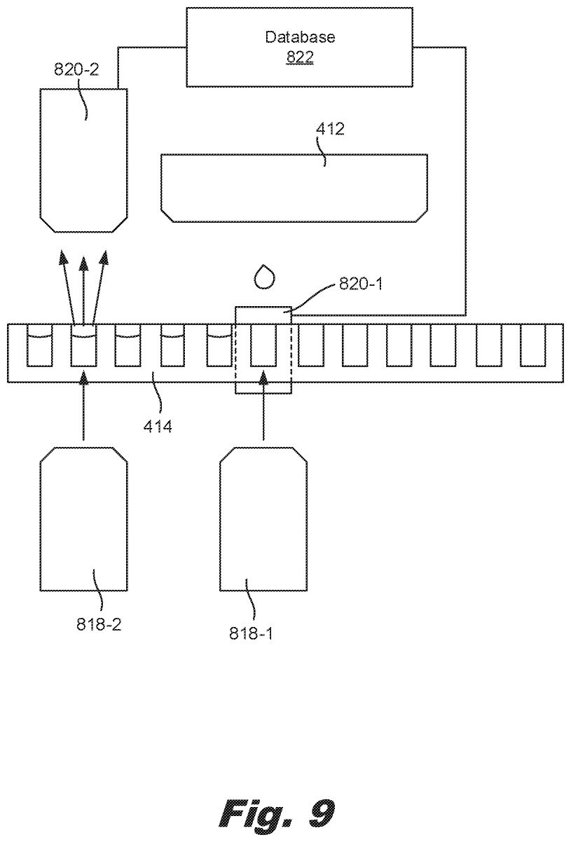

FIG. 9 is a diagram of a particle dispensing system (FIG. 1, 100) with optical particle sensing following fluid ejection, according to an example of the principles described herein. As depicted in FIG. 8, the light source (818) and the detector (820) may be colinear. However, in other examples, the light source (818) and the detector (820) may be perpendicular to one another. An example of such a system is depicted in FIG. 9 where the first light source (818-1) emits light, and a detector (820-1) is perpendicular to the first light source (818-1). As described above, the cells in the droplet scatter light in all directions. In this example, the detector (820-1) captures light that scatters to the side, i.e., into the page. Such a side-scatter detection system may allow for the light source (818-1) to be directly underneath the fluid ejector (412) such that light-scattering cell counting is performed as the fluid droplet is being ejected.

Also as described above, the optical verification system (FIG. 1, 104) may be oriented in different ways. FIG. 8 depicted an example where the optical verification system (FIG. 1, 104) is directed across a path of the fluid as it falls onto the substrate (414). However, as depicted in FIG. 9, the light source (818) and the detector (820) may be directed upward through the substrate (414).

In another example, the light source (818) and the detector (820) may be directed downward through the substrate (414). That is, rather than having the second detector (820-2) above the substrate (414) and the second light source (818-2) below the substrate (414) as depicted in FIG. 9, the light source (818) may be above the substrate (414) and the detector (820) may be below the substrate (414).

FIG. 9 also depicts an example where the optical verification system (FIG. 1, 104) includes multiple optical verification devices, i.e., multiple light source (818-1, 818-2)/detector (820-1, 820-2) pairs. As with multiple cameras (FIG. 4, 416), multiple light source (818)/detector (820) pairs may allow for more accurate verification of single-cell ejection as the output of the multiple detectors (820) may be compared to one another.

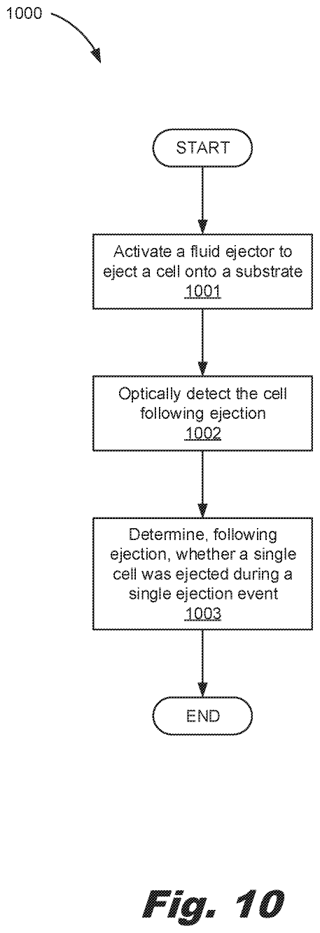

FIG. 10 is a flow chart of a method (1000) of optical particle sensing following fluid ejection, according to an example of the principles described herein. According to the method (1000), a fluid ejector (FIG. 4, 412) is activated (block 1001) to eject a cell onto a substrate (FIG. 4, 414). That is, as described above, the particle dispensing system (FIG. 1, 100) may include a controller (FIG. 1, 106) which sends an electrical signal to an installed fluid cartridge to activate a fluid ejector (FIG. 4, 412), which fluid ejector (FIG. 4, 412) operates to eject a small amount of fluid onto a target surface.

Following ejection, the fluid that was ejected is optically detected (block 1002). That is, an optical verification system (FIG. 1, 104) such as one or multiple cameras (FIG. 4, 416) or one or multiple light source (FIG. 8, 818)/detector (820) pairs track what was ejected. Such tracking may be either while the fluid is in flight from the fluid ejector (FIG. 4, 412) to the substrate (FIG. 4, 414), or may be after the fluid has been deposited in a well as described above.

Following ejection, it is then determined (block 1003) whether a single cell was ejected during the ejection event. That is, the optical verification system (FIG. 1, 104), in whatever form, may be able to distinguish between a single cell and multiple cells and therefore verifies whether a single cell was ejected or more generally counts the number of cells ejected. Note that such a determination (block 1003) occurs after the ejection event. That is, rather than verifying before ejection that a single cell is expected to be ejected, the current method (1000) verifies that what was actually ejected was a single cell. Doing so may be more accurate than pre-dispense systems as any number of scenarios may alter what happens between a pre-dispense sensing of a single cell and an ejection of that single cell. For example, after a sensor detects multiple cells, but before they are ejected, cells may accumulate in a fluid path or in the fluid ejector (FIG. 4, 414) itself.

As described above, activation (block 1001) of a fluid ejector (FIG. 4, 412) may occur over a first location of the substrate (FIG. 4, 414) while determining whether a single cell was ejected during the ejection event occurs over a second location of the substrate (FIG. 4, 414). That is, the system may simultaneously eject cells into wells while verifying that single cells have been ejected into other cells. Such parallel operation increases efficiency as multiple operations are carried out at the same time. In some examples, the simultaneous activation (block 1001) and ejection verification may occur at a single site as described above. For example, as depicted in FIG. 5, a camera (FIG. 4, 416) or other optical verification system (FIG. 1, 104) may operate directly under a fluid ejector (FIG. 4, 412).

In some examples, determining (block 1003) whether a single cell was ejected during an ejection event is triggered by an ejection monitoring system which indicates uncertainty regarding a quantity of cells ejected. That is, in some examples, the particle dispensing system (FIG. 1, 100) may include a cell dispense sensor system that monitors and/or counts cells prior to ejection. In an example, the cell dispense sensor system may include an impedance sensor within a microfluidic path towards the fluid ejector. Such a cell dispense sensor system may indicate whether a single cell is present in a portion of fluid headed towards the fluid ejector. Such a cell dispense sensor system is a pre-dispense sensor. However, it may be the case that due to user error, characteristics of the fluid being ejected, or any number of reasons, the cell dispense sensor system may be unsure whether a target number of cells has been ejected. When this condition occurs, the particle dispensing system (FIG. 1, 100) may trigger the optical verification system (FIG. 1, 104) to verify the amount ejected. In this example, the optical verification system (FIG. 1, 104) may not operate for every ejection event, just those instances where the confidence is below a threshold from an upstream sensor.

In summary, using such a particle dispensing system 1) allows single cell sorting of a sample; 2) uses fluid ejection to separate cells from carrier fluid; 3) separates fluid pumping and fluid ejecting; 4) increases number of wells with single cells of a desired type; and 5) provides highly accurate cell separation and identification. However, the devices disclosed herein may address other matters and deficiencies in a number of technical areas.

1. A particle dispensing system, comprising:
a port to receive a number of fluid cartridges, each fluid cartridge to hold an amount of fluid, the fluid comprising particles to be ejected;
an optical verification system to determine, following ejection, a count of a number of particles ejected during an ejection event; and
a controller to selectively activate a number of fluid ejectors to eject the amount of fluid.

2. The particle dispensing system of claim 1, wherein the particles within the fluid comprise cells to be ejected.

3. The particle dispensing system of claim 1, wherein the optical verification system is selected from the group consisting of:
a camera; and
a light source to emit light and a detector to capture scattered light from the light source.

4. The particle dispensing system of claim 3, further comprising a database to map detected scattered light to the count of the number of particles.

5. The particle dispensing system of claim 1, wherein the optical verification system is directed downward towards a substrate on which the fluid is deposited.

6. The particle dispensing system of claim 1, wherein the optical verification system is directed upward through a substrate on which the fluid is deposited.

7. The particle dispensing system of claim 1, wherein the optical verification system is directed across a path of the fluid as it falls onto the substrate.

8. A method, comprising:
activating a fluid ejector of a particle dispensing system to eject a cell onto a substrate;
optically detecting the fluid following ejection; and
determining, following ejection and with an optical verification system, whether a desired quantity of cells was ejected during an ejection event.

9. The method of claim 8, wherein determining whether the desired quantity of cells was ejected during the ejection event is triggered by an ejection monitoring system indicating uncertainty regarding a quantity of cells ejected.

10. The method of claim 8, wherein optically detecting the fluid following ejection comprises optically detecting the fluid as it falls.

11. The method of claim 8, wherein optically detecting the fluid following ejection comprises optically detecting the fluid on the substrate.

12. The method of claim 8, wherein as fluid is being deposited in a first location of the substrate, an optical verification system is determining whether a single cell was ejected at a second location previously dispensed onto.

13. A particle dispensing system, comprising:
a port to receive a number of fluid cartridges, each fluid cartridge to hold cells to be ejected individually during an ejection event;
an optical verification system to, following ejection, verify that a single cell was ejected during the ejection event;
a controller to selectively activate a number of fluid ejectors to eject the amount of fluid; and
a stage controller to suppress a motion of a stage holding a substrate during optical verification.

14. The particle dispensing system of claim 13, wherein the optical verification system comprises multiple optical verification devices to verify that the single cell was ejected during the ejection event.

15. The particle dispensing system of claim 13, further comprising the stage to hold the substrate, wherein the stage allows optical verification from underneath the substrate.
